(19) United States
(12) Patent Application Publication
(10) Pub. No.: US 2007/0048232 A1
(43) Pub. Date: Mar. 1, 2007

(54) Composition containing at least 50% by weight of dyestuffs

(75) Inventor: Fabienne Bouchard, Villejuif (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/508,863

(22) Filed: Aug. 24, 2006

Related U.S. Application Data
(60) Provisional application No. 60/712,427, filed on Aug. 31, 2005.

(30) Foreign Application Priority Data
Aug. 26, 2005 (FR) 05 52575

(51) Int. Cl. C09B 63/00; C08K 5/00; C09B 67/00; C11C 3/00; A61K 8/81

(52) U.S. Cl. 424/59; 106/504; 106/493; 106/402; 106/502; 424/70.7

(57) ABSTRACT

The invention relates to an eyeshadow composition containing at least 50% by weight, relative to the total weight of the composition, of at least one colouring agent that produces a colour by absorption of at least part of the visible spectrum.

### REFERENCE TO PRIOR APPLICATIONS

This application claims priority to U.S. provisional application 60/712,427 filed Aug. 31, 2005, and to French patent application 0552575 filed Aug. 26, 2005, both incorporated herein by reference.

### FIELD OF THE INVENTION

The present invention relates to a composition comprising a high content of dyestuffs. Preferably, the invention composition is a cosmetic eyeshadow. The invention also relates to a process for making up the eyelids using the composition.

In a preferred embodiment the invention relates to an eyeshadow composition comprising at least 50% by weight, relative to the total weight of the composition, of at least one colouring agent that produces a colour by absorption of at least part of the visible spectrum.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. AS will be realized, the present invention is capable of other and different embodiments, and its several details are capable of modifications in various obvious respects, all without departing from the present intention. The description is to be regarded as illustrative in nature, and not as restrictive.

### BACKGROUND OF THE INVENTION

Eyeshadows generally contain a suitable vehicle and of dyestuffs intended to give a certain colour to the makeup product and, after application, to the eyelids. They may be in water-based or anhydrous form.

Many makeup compositions of this type are in compact powder form generally comprising a fatty phase, conventionally known as a “binder”, and a pulverulent phase especially comprising pigments and/or fillers. The main function of the binder is to ensure sufficient cohesion of the final composition, especially so as to prevent it from fragmenting, as may be caused by impacts, and moreover to make it easy to be taken up.

Obtaining these properties moreover implies that the other components of the cosmetic composition, and in particular the fillers they contain, also prove to be suitable for a formulation of compact powder type.

It is sought to obtain eyeshadows that have a strong colour effect, in particular by incorporating a high content of dyestuffs such as mineral or organic pigments.

However, conventional pigments, for instance iron oxides, show good compactability, whi ch may be reflected by excessive cohesion of the product, making its erosion difficult using an applicator such as a powder puff or a brush, or even by finger.

Consequently, the incorporation of a high content of dyestuff is limited by the requirement of maintaining a good capacity for uptake of the product.

### OBJECTS OF THE INVENTION

One aim of the present invention is to provide a composition, preferably useful as an eyeshadow, in the form of a compact powder comprising a high content of dyestuffs and having a strong colour, good cohesion, good impact strength and a good uptake capacity.

### DETAILED DESCRIPTION OF THE PREFERRED EMBODIMENTS

One preferred embodiment of the present invention is a composition, preferably useful as an eyeshadow, in compact powder form comprising at least 50% by weight, relative to the total weight of the composition, of at least one colouring agent that produces a colour by absorption of at least part of the visible spectrum and a solid fatty phase comprising at least one compound chosen from waxes.

A colour produced by absorption of light is also occasionally termed a chemical colour, as opposed to the colours produced by an interference phenomenon, including diffraction, which are also referred to as physical colours. The phenomenon of absorption of light energy by the colouring agent may be based on electronic transitions.

A subject of the invention is also the use, in a composition, preferably useful as an eyeshadow, of at least 50% by weight, relative to the total weight of the composition, of at least one colouring agent that produces a colour by absorption of at least part of the visible spectrum, to obtain a composition, preferably useful as an eyeshadow, that has a strong colour, good cohesion, good impact strength and a good uptake capacity.

A subject of the invention is also a cosmetic process for making up the eyelids, comprising the application to the eyelids of the composition/eyeshadow defined above.

As noted, the invention pertains generally to compositions. However, the preferred form of the invention compositions are eyeshadows, and the preferred uses of the invention composition are those uses common to eyeshadows. Thus, for brevity, the invention compositions described herein below are referred to as eyeshadows. However, the invention is not so limited.

Colouring Agent

The colouring agent that produces a colour by an absorption phenomenon is not limited and includes any of an organic or mineral or hybrid pigment comprising both organic material and mineral material. Mixtures may be used.

The colouring agent may be a particulate or non-particulate compound.

When the colouring agent comprises a dye, this dye may be chosen from liposoluble and water-soluble dyes.

The liposoluble dyes include, for example, Sudan red, DC Red 17, DC Green 6, β-carotene, soybean oil, Sudan brown, DC Yellow 11, DC Violet 2, DC Orange 5 and quinoline yellow.

The water-soluble dyes include, for example, beetroot juice and methylene blue.

The amount of dyes are not limited and may represent, for example, from 0.1% to 20% or even from 0.1% to 6% of the weight of the composition, when present.

The colouring agent may also be an organic pigment or lake chosen from the materials below, and mixtures thereof:
cochineal carmine,
organic pigments of azo, anthraquinone, indigoid, xanthene, pyrene, quinoline, triphenylmethane or fluorane dyes,
organic lakes or insoluble sodium, potassium, calcium, barium, aluminium, zirconium, strontium or titanium salts of acidic dyes such as azo, anthraquinone, indigoid, xanthene, pyrene, quinoline, triphenylmethane or fluorane dyes, these dyes possibly comprising at least one carboxylic or sulfonic acid group.

Included among the organic pigments that may especially be mentioned are the D & C certified pigments known under the following names: D&C Blue No. 4, D&C Brown No. 1, D&C Green No. 5, D&C Green No. 6, D&C Orange No. 4, D&C Orange. No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red. No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, FD&C Yellow No. 6.

The Organic dyestuff may be an organic lake supported on an organic support such as rosin or aluminium benzoate, for example.

Included among the organic lakes that may be mentioned in particular are the D & C certified pigments known under the following names: D&C Red No. 2 Aluminium lake, D&C Red No. 3 Aluminium lake, D&C Red No. 4 Aluminium lake, D&C Red No. 6 Aluminium lake, D&C Red No. 6 Barium lake, D&C Red No. 6 Barium/Strontium lake, D&C Red No. 6 Strontium lake, D&C Red No. 6 Potassium lake, D&C Red No. 7 Aluminium lake, D&C Red No. 7 Barium lake, D&C Red No. 7 Calcium lake, D &C Red No. 7 Calcium/Strontium lake, D&C Red No. 7 Zirconium lake, D&C Red No. 8 Sodium lake, D&C Red No. 9 Aluminium lake, D&C Red No. 9 Barium lake, D&C Red No. 9 Barium/Strontium lake, D&C Red No. 9 Zirconium lake, D&C Red No. 10 Sodium lake, D&C Red No. 19 Aluminium lake, D&C Red No. 19 Barium lake, D&C Red No. 19 Zirconium lake, D&C Red No. 21 Aluminium lake, D&C Red No. 21 Zirconium lake, D&C Red No. 22 Aluminium lake, D&C Red No. 27 Aluminium lake, D&C Red No. 27 Aluminium/Titanium/Zirconium lake, D&C Red No. 27 Barium lake, D&C Red No. 27 Calcium lake, D&C Red No. 27 Zirconium lake, D&C Red No. 28 Aluminium lake, D&C Red No. 30 lake, D&C Red. No. 31 Calcium lake, D&C Red No. 33 Aluminium lake, D&C Red No. 34 Calcium lake, D&C Red No. 36 lake, D&C Red No. 40 Aluminium lake, D&C Blue No. 1 Aluminium lake, D&C Green No. 3 Aluminium lake, D&C Orange No. 4 Aluminium lake, D&C Orange No. 5 Aluminium lake, D&C Orange No. 5 Zirconium lake, D&C Orange No. 10 Aluminium lake, D&C Orange No. 17 Barium lake, D&C Yellow No. 5 Aluminium lake, D&C Yellow No. 5 Zirconium lake, D&C Yellow No. 6 Aluminium lake, D&C Yellow No. 7 Zirconium lake, D&C Yellow No. 10 Aluminium lake, FD&C Blue No. 1 Aluminium lake, FD&C Red No. 4 Aluminium lake, FD&C Red No. 40 Aluminium lake, FD&C Yellow No. 5 Aluminium lake, FD&C Yellow No. 6 Aluminium lake.

The chemical materials corresponding to each of the organic dyestuffs mentioned above are mentioned in the publication “International Cosmetic Ingredient Dictionary and Handbook”, 1997 edition, pages 371 to 386 and 524 to 528, published by “The Cosmetic, Toiletry, and Fragrance Association”, the content of which is incorporated into the present patent application by reference.

The colouring agent may comprise a composite pigment, comprising a core at least partially coated with a shell.

Composite Pigments

A composite pigment according to the invention includes those that may be composed especially of particles comprising:
a mineral core,
at least one at least partial coating of at least one organic dyestuff.

At least one binder may advantageously contribute to the fixing of the organic dyestuff to the mineral core.

The composite pigment particles may have varied forms. These particles may especially be in platelet or globular form, in particular spherical, and may be hollow or solid. The term “platelet form” denotes particles for which the ratio of the largest size to the thickness is greater than or equal to 5

A composite pigment may have, for example, a specific surface area of between 1 and 1000 m²/g, especially between 10 and 600 m²/g approximately and in particular between 20 and 400 m²/g approximately. The specific surface area is the value measured by the BET method.

The mass proportion of the core may exceed 50% and may range, for example, from 50% to 70%, for example from 60% to 70%, relative to the total weight of the composite pigment.

The composite pigment may be different from an interference pigment as described, for example, in U.S. Pat. No. 6,428,773. An interference pigment comprises, for example, several layers of constant thicknesses of materials selected to be able to produce optical interferences.

The saturation C* of the composite pigment may be greater than or equal to 30, measured according to the protocol below.

Protocol for Measuring the Saturation of the Composite Pigment:

The values a* and b* in the CIE L*a*b* space of the composite pigment are measured as follows:

The pure composite pigment is compacted in a 2×1.5 cm rectangular dish 3 mm deep, by applying a pressure of 100 bar. The values a* and b* of the compacted pigment are measured with a Minolta 3700d spectrophotometer, in excluded specular mode, under illuminant D65, with medium aperture. The saturation is given by C*=(a*²+b*²) ^{1/2}

Mineral Core

The mineral core may be in any form that is suitable for fixing organic pigment particles, for example spherical, globular, granular, polyhedral, acicular, fusiform, in the form of flakes, grains of rice or scales, and also a combination of these forms, this list not being limiting.

Preferably, the ratio of the largest dimension to the smallest dimension is between 1 and 50.

The mineral core may have a mean size of between about 1 nm and about 100 nm, or even between about 5 nm and about 75 nm, for example between about 10 nm and about 50 nm and especially 20 or 25 nm.

The term “mean size” denotes the dimension given by the statistical particle size distribution to half the population, which is denoted the D50. The mean size may be a number-average size determined by image analysis (electron microscopy).

The mineral core may have a refractive index of greater than or equal to 2, or even greater than or equal to 2.1, for example greater than or equal to 2.2.

The mineral core may for example be made of a magnetic or non-magnetic material chosen from the non-limiting list comprising metal salts and metal oxides, especially titanium oxides, zirconium oxides, cerium oxides, zinc oxides, iron oxides, ferric blue, aluminium oxides and chromium oxides, aluminas, glasses, ceramics, graphite, silicas, silicates, especially aluminosilicates and borosilicates, and synthetic mica, and mixtures thereof.

Titanium oxides, especially TiO₂, iron oxides, especially Fe₂O₃, cerium oxides, zinc oxides and aluminium oxides, silicates, especially alumino-silicates and borosilicates, are most particularly suitable.

The mineral core may have a specific surface area, measured by the BET method, of between, for example, about 1 m²/g and about 1000 m²/g and better still between about 10 m²/g and about 600 m²/g, for example between about 20 m²/g and about 400 m²/g.

Where appropriate, the mineral core may be coloured.

Organic Dyestuff

The organic dyestuff may comprise, for example, at least one organic pigment, for example at least one organic lake.

The organic dyestuff may be chosen, for example, from particulate compounds that are insoluble in the physiologically acceptable medium of the composition.

The organic dyestuff may comprise, for example, pigments, for example organic lakes or other organic dyestuffs, which may be chosen from the organic lakes and pigments mentioned above.

The mass proportion of organic dyestuff may be between about 10 parts and about 500 parts by weight per 100 parts of the mineral core, or even between about 20 parts and about 250 parts by weight, for example between about 40 parts and about 125 parts by weight per 100 parts of the mineral core.

The proportion of organic dyestuff is not limited and may exceed 30%, for example it may range from 30% to 50% and, for example, from 30% to 40%, relative to the total weight of the composite pigment.

Binder of the Composite Pigment

The binder of the composite pigment may be of any type provided that it allows the organic dyestuff to adhere to the surface of the mineral core.

The binder may be chosen especially from a non-limiting list comprising silicone compounds, polymeric or oligomeric compounds or the like, and in particular from organosilanes, fluoroalkyl organosilanes and polysiloxanes, for example polymethylhydrogensiloxane, and also various couplers, such as couplers based on silanes, on titanates, on aluminates or on zirconates, and mixtures thereof.

The silicone compound may be chosen from a non-limiting list especially comprising:
the organosilanes (1) obtained from alkoxysilanes,
the modified or unmodified polysiloxanes (2) chosen from a non-limiting list comprising:
the modified polysiloxanes (2A) comprising at least one radical chosen especially from polyethers, polyesters and epoxy compounds (these will be referred to as modified polysiloxanes”),
the polysiloxanes (2B) bearing, on a silicon atom located at the end of the polymer, at least one group chosen from a non-limiting list comprising carboxylic acids, alcohols and hydroxyl groups, and
the fluoroalkyl organosilane compounds (3) obtained from fluoroalkylsilanes.

The compounds serving as binder may especially have a molar mass that may range between 300 and 100 000.

To obtain a layer that covers the mineral cores uniformly, the binder is preferably in liquid form or in a form soluble in water or in various solvents.

The amount of binder is not limited and may preferably range from 0.01% to 15%, especially from 0.02% to 12.5% and in particular from 0.03% to 10% by weight (calculated relative to C or Si) relative to the weight of the particles comprising the core and the binder. For further details regarding the manner of calculating the relative amount of the binder, reference may be made to patent application EP 1 184 426 A2. The relative proportion of binder may be less than or equal to 5%, for example less than or equal to 3%, relative to the total weight of the composite pigment.

The composite pigment may be made, for example, via one of the processes described in European patent applications EP 1 184 426 and EP 1 217 046, the content of which is incorporated herein by reference, advantageously via the process described in patent application EP 1 184 426.

Another method for manufacturing a composite pigment is described in patent JP 3286463, which discloses a solution precipitation process.

Preferably, the colouring agent is chosen from:

organic pigments such as

carbon black,
the pigments certified D&C by the Food & Drug Administration as listed in the section “Color Additives—Batch Certified by the U.S. Food and Drug Administration” of the CTFA; mention may be made especially of Blue 1 and 4, Brown 1, Ext. Violet 2, Ext. Yellow 7, Green 3, 5, 6 and 8, Orange 4, 5, 10 and 11, Red 4, 6, 7, 17, 21, 22, 27, 28, 30, 36 and 40, Violet 2, Yellow 5, 6, 7, 8, 10 and 11,
lakes based on cochineal carmine or on barium, strontium, calcium or aluminium,

mineral pigments such as:
iron oxide, titanium oxide, zirconium oxide, cerium oxide, zinc oxide, iron oxide or chromium oxide,
ferric blue, manganese violet, ultramarine blue, pink or violet, chromium hydrate, chromium hydroxide or bismuth oxychloride,
and mixtures thereof.

The eyeshadow according to the invention preferably comprises the dyestuff(s) in a content of greater than or equal to 50% by weight, for example ranging from 50% to 90% by weight, preferably in a content of greater than or equal to 55% by weight, for example ranging from 55% to 80% by weight and better still greater than or equal to 60% by weight, for example from 60% to 70%, relative to the total weight of the composition, including 51, 53, 57, 59, 61, 63, 65, 67, 69, 71, 73, 75, 77 , 79, 81, 83, 85, 87, 89, 91, etc. % relative to the total weight of the composition including all values and subranges between stated values.

Solid Fatty Phase:

The composition according to the invention advantageously comprises at least one solid fatty phase, also known as a “dry binder or solid binder”. The solid fatty phase comprises at least one compound chosen from waxes.

For the purposes of the present invention, the term “solid binder” means a fatty phase whose melting point is greater than or equal to 30° C and may especially range from 30 to 250° C and in particular from 30 to 230° C. Preferred melting points include those greater than or equal to 40, 50, 60, 70, 80, 90, 100, 110, 120, etc. ° C. including all values and subranges between stated values.

For the purposes of the present invention, the term “wax” means a lipophilic fatty compound, which is solid at room temperature (25°C.) and atmospheric pressure (760 mmHg, i.e. 105 Pa) which undergoes a reversible solid/liquid change of state, and which in particular has a melting point of greater than or equal to 30° C., especially greater than or equal to 55° C., which may be up to 250° C., especially up to 230° C. and in particular up to 120° C.

By bringing the wax to its melting point, it is possible to make it miscible with the oils and to form a microscopically homogeneous mixture, but on returning the temperature of the mixture to room temperature, recrystallization of the wax in the oils of the mixture is obtained.

According to the invention, the melting point values correspond to the melting peak measured using a differential scanning calorimeter (DSC), for example the calorimeter sold under the name DSC 30 by the company Mettler, with a temperature rise of 5 or 10° C. per minute.

For the purposes of the invention, the waxes include those generally used in cosmetics or dermatology. They may especially be hydrocarbon-based waxes, silicone waxes and/or fluoro waxes, optionally comprising ester or hydroxyl functions. They may also be of natural or synthetic origin.

Non-limiting illustrations of these waxes that may especially be mentioned include:
beeswax, lanolin wax and Chinese insect waxes; rice wax, carnauba wax, candelilla wax, ouricury wax, cork fibre wax, sugarcane wax, Japan wax and sumach wax; montan wax; microcrystalline waxes, paraffin waxes, ozokerites, ceresin wax, lignite wax, polyethylene waxes, the waxes obtained by Fisher-Tropsch synthesis, and fatty acid esters and glycerides that are solid at 40° C. and especially above 55° C.,
the waxes obtained by catalytic hydrogenation of animal or plant oils containing linear or branched C₈-C₃₂ fatty chains; especially hydrogenated jojoba oil, hydrogenated sunflower oil, hydrogenated castor oil, hydrogenated coconut oil and hydrogenated lanolin oil,
silicone waxes or fluoro waxes, and
mixtures thereof.

Preferably, the solid fatty phase comprises at least one wax that is totally or partially in powder form and especially in micronized form.

Preferably, the wax is chosen from carnauba waxes and paraffin waxes, and mixtures thereof.

Among the waxes that may be used in powder form, mention may be made especially of the carnauba wax microbeads sold under the name MicroCare 350® by the company Micro Powders and the paraffin wax microbeads sold under the name MicroEase 114S® by the company Micro Powders.

The Solid fatty phase may comprise at least one compound chosen from metal soaps.

Among these, mention may be made especially of the metal soaps of fatty acids containing from 12 to 22 carbon atoms and in particular those containing from 12 to 18 carbon atoms.

The metal of the metal soap may especially be zinc or magnesium.

The fatty acid may especially be chosen from lauric acid, myristic acid, stearic acid and palmitic acid.

Metal soaps that may be used include zinc laurate, magnesium stearate, magnesium myristate and zinc stearate, and mixtures thereof.

According to one particular variant of the invention, the Solid fatty phase may comprise at least one metal soap that is totally or partially in powder form.

The solid fatty phase may be present in any content, preferably one ranging from 1% to 30% by weight and especially from 2% to 20% by weight relative to the total weight of the composition, including 5, 8, 10, 12, 15, and 18% by weight including all values and subranges between stated values.

Liquid Fatty Phase:

The composition according to the invention may also comprise a liquid fatty phase (also known as a fatty binder) comprising at least one oil.

This oil includes oils conventionally used as binder in compact powders.

Invention oils may especially be chosen from:
soybean oil, grapeseed oil, sesame seed oil, corn oil, rapeseed oil, sunflower oil, cotton seed oil, avocado oil, olive oil, castor oil, jojoba oil and groundnut oil;
hydrocarbon oils such as liquid paraffins, squalane, petroleum jelly and polydecene, especially hydrogenated polydecene, for instance “Ceraflow E®” sold by the company Shamrock;
fatty esters, such as isopropyl myristate, isopropyl palmitate, butyl stearate, isodecyl stearate, hexyl laurate, isononyl isononanoate, 2-ethylhexyl palmitate, 2-hexyldecyl laurate, 2-octyldecyl palmitate, 2-octyldodecyl myristate or lactate, 2-diethylhexyl succinate, diisostearyl malate, and glyceryl or diglyceryl triisostearate;
silicone oils such as polymethylsiloxanes, polymethylphenylsiloxanes, polysiloxanes modified with fatty acids, fatty alcohols or polyoxyalkylenes, fluoro silicones or perfluoro oils;
higher fatty acids such as myristic acid, palmitic acid, stearic acid, behenic acid, oleic acid, linoleic acid, linolenic acid or isostearic acid;
higher fatty alcohols such as cetanol, stearyl alcohol or oleyl alcohol;
mixtures thereof.

According to one advantageous embodiment, the eyeshadow according to the invention comprises a liquid fatty phase in a content of less than or equal to 10% by weight, preferably less than or equal to 7% by weight, preferably less than or equal to 5% by weight, better still less than or equal to 3% by weight and even better still less than or equal to 3% by weight, for example less than or equal to 2.8, 2.6, 2.4, 2.2, 2, 1.8, 1.6, 1.4, 1.2, 1, 0.8, 0.6, etc. % by weight relative to the total weight of the composition; even better still, the eyeshadow composition is free of liquid fatty phase. This makes it possible to limit the “waxing” phenomena: the product is said to wax when it is too compact and difficult to take up.

Fillers

The eyeshadow according to the invention may comprise at least one filler that for example may be organic or mineral, and of spherical or lamellar form.

The fillers may be chosen from incompactable fillers such as:
silica microspheres, especially of open porosity or, preferably, hollow silica microspheres, such as the products “Silica Beads SB 700/HA” or “Silica Beads SB 700” from the company Maprecos; these microspheres may be impregnated with a cosmetic active agent;
microporous polymer microspheres, which have a structure similar to that of a sponge; they generally have a specific surface area of at least 0.5 m²/g and in particular of at least 1 m²/g, the said specific surface area having no upper limit other than that resulting from the practical possibility of making microspheres of very high porosity: the specific surface area may be, for example, up to 1000 m²/g or even more. Illustrations of these microspheres that may be mentioned include acrylic polymer microspheres, such as those made of crosslinked acrylate copolymer “Polytrap 6603 Adsorber” from the company RP Scherer, and those made of polymethyl methacrylate Micropearl M 100 from the company SEPPIC;
polyurethane powder, such as the powdered copolymer of hexamethylene diisocyanate and of trimethylol hexyl lactone sold under the names Plastic Powder D-400 and T-7 by the company Toshiki;
polymer microcapsules bearing only one closed cavity and form a reservoir, which may contain a liquid, especially a cosmetic active agent; they are prepared via known processes, such as those described in patents U.S. Pat. No. 3,615,972 and EP-A 0 56 219. They may be made, for example, of polymers or copolymers of ethylenically unsaturated acid, amine or ester monomers, of urea-formaldehyde polymers or of vinylidene chloride polymers or copolymers; by way of example, mention may be made of microcapsules made of methyl acrylate or methacrylate polymers or copolymers, or alternatively of copolymers of vinylidene chloride and of acrylonitrile; among these polymers, mention will be made especially of those containing 20-60% by weight of units derived from vinylidene chloride, 20-60% by weight of units derived from acrylonitrile and 0-40% by weight of other units such as units derived from an acrylic and/or styrene monomer; crosslinked acrylic polymers or copolymers may also be used;
elastomeric crosslinked organopolysiloxane spherical powders, described especially in document JP-A-02 243 612, such as those sold under the name “Trefil Powder E-506C” by the company Dow Corning.

The composition may also contain, alone or in addition to the incompactable fillers, compactable fillers.

Compactable fillers of lamellar form that may be mentioned include:
talcs or hydrated magnesium silicates, especially in the form of particles generally less than 40 μm in size;
micas or aluminosilicates of varied composition that are especially in the form of flakes from 2 to 200 μm and preferably 5-70 μm in size and from 0.1 to 5 μm and preferably 0.2-3 μm in thickness, these micas possibly being of natural origin (for example muscovite, margarite, roscoelite, lipidolite or biotite) or of synthetic origin;
clays such as sericites, which belong to the same chemical and crystalline class as muscovite;
kaolin or hydrated aluminium silicate, which is especially in the form of particles of isotropic forms generally less than 30 μm in size;
boron nitrides;
powders of tetrafluoroethylene polymers, such as “Ceridust 9205 F” from the company Clariant;
precipitated calcium carbonate, especially in the form of particles greater than 10 μm in size;
magnesium carbonate and magnesium hydrogen carbonate;
hydroxyapatite;
powders of non-expanded synthetic polymers, such as polyethylene, polyesters (for example polyethylene isophthalate or terephthalate) and polyamides (for example Nylon), in the form of particles less than 50 μm in size;
powders of spheronized, crosslinked or non-crosslinked synthetic polymers, for instance polyamide powders such as poly-β-alanine powder or Nylon powder, for example Orgasol powder from the company Atochem, polyacrylic acid or polymethacrylic acid powder, powders of polystyrene crosslinked with divinylbenzene, and silicone resin powders, and
Powders of organic materials of natural origin, for instance starches, especially corn starch, wheat starch or rice starch;
   and mixtures thereof.

The fillers may be present in any content, including for example a content ranging from 0.1% to 50% by weight and preferably ranging from 1% to 40% by weight relative to the total weight of the composition.

The eyeshadow according to the invention may comprise, besides the colouring agents mentioned above, an additional colouring agent, chosen from nacreous pigments, which may be chosen from white nacreous pigments such as mica coated with titanium or with bismuth oxychloride, coloured nacreous pigments such as titanium mica with iron oxides, titanium mica with, especially, ferric blue or chromium oxide, titanium mica with an organic pigment of the abovementioned type, and also nacreous pigments based on bismuth oxychloride.

The composition may comprise at least one interference or diffractive pigment, and/or reflective particles.

Mention may be made of pigments with an effect such as particles comprising a natural or synthetic, organic or mineral substrate, for example glass, ceramics, aluminas, silicas, silicates and especially aluminosilicates and borosilicates, synthetic mica such as fluorophlogopite, acrylic resins, polyester, polyurethane, polyethylene terephthalate, metals and metal derivatives, and mixtures thereof, this list not being limiting, the said substrate being optionally coated with metal substances, for instance aluminium, gold, silver, platinum, copper or bronze, or metal oxides, for instance titanium dioxide, iron oxide or chromium oxide, and mixtures thereof.

Goniochromatic colouring agents, interference pigments, especially liquid-crystal or multilayer interference pigments, thermochromic agents and optical brighteners may also be used.

The composition according to the invention may be an anhydrous composition, i.e. a composition containing less than 2% by weight of water, or even less than 0.5% by weight of water, especially less than 0.2% water, the water not being added during the preparation of the composition, but corresponding to the residual water provided by the mixed ingredients.

The composition may comprise other ingredients (adjuvants), such as preserving agents, cosmetic active agents, softeners, UV-screening agents, thickeners, surfactants and fragrances.

Needless to say, a person skilled in the art will take care to select the optional adjuvent(s) added to the composition according to the invention such that the advantageous properties intrinsically associated with the composition according to the invention are not, or are not substantially, adversely affected by the envisaged addition.

The composition may be prepared by mixing together the ingredients of the pulverulent phase (fillers and pigments), followed by adding the fatty phase with stirring, the mixture then being ground and/or mixed vigorously, optionally screened, and then poured into a dish and compacted, for example using a press.

The composition thus obtained is in the form of a compact powder.

The present invention is illustrated in greater detail in the non-limiting examples that follow:

### EXAMPLE 1

The following compacted eyeshadow was prepared:

| | | |
|---|---|---|
| Black iron oxides | 60 | g |
| Ultramarines and silica | 0.5 | g |
| Talc | 25.7 | g |
| Magnesium stearate | 8 | g |
| Paraffin wax microbeads | 5 | g |
| MicroEase ® 114S from Micro Powders) | | |
| Hydrogenated polydecene | 3.00 | g |
| (Ceraflow E ® from Shamrock) | | |
| Preserving agents | 0.8 | g |

The compacted composition shows good impact strength and is easy to erode using an applicator.

The above written description of the invention provides a manner and process of making and using it such that any person skilled in this art is enabled to make and use the same, this enablement being provided in particular for the subject matter of the appended claims, which make up a part of the original description and including a composition, preferably in the form of an eyeshadow, in compact powder form comprising at least 50% by weight, relative to the total weight of the composition, of at least one colouring agent that produces a colour by absorption of at least part of the visible spectrum and a solid fatty phase comprising at least one compound chosen from waxes.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition in compact powder form comprising at least 50% by weight, relative to the total weight of the composition, of at least one colouring agent that produces a colour by absorption of at least part of the visible spectrum and a solid fatty phase comprising at least one wax.

2. The composition according to claim 1, wherein the colouring agent is present in a content of from 50% to 90% by weight relative to the total weight of the composition.

3. The composition according to claim 1, wherein the colouring agent is present in a content of greater than or equal to 55% by weight relative to the total weight of the composition.

4. The composition according to claim 1, wherein the colouring agent is chosen from:
organic pigments such as
carbon black,
Blue 1 and 4, Brown 1, Ext. Violet 2, Ext. Yellow 7, Green 3, 5, 6 and 8, Orange 4, 5, 10 and 11, Red 4, 6, 7, 17, 21, 22, 27, 28, 30, 36 and 40, Violet 2, Yellow 5, 6, 7, 8, 10 and 11,
lakes based on cochineal carmine or on barium, strontium, calcium or aluminium,
mineral pigments , and
mixtures thereof.

5. The composition according to claim 1, wherein the solid fatty phase further comprises at least one metal soap.

6. The composition according to claim 1, wherein the wax is totally or partially in powder form.

7. The composition according to claim 1, comprising at least one of a paraffin wax and a carnauba wax.

8. The composition according to claim 1, wherein the solid fatty phase represents from 1% to 30% by weight relative to the total weight of the composition.

9. The composition according to claim 1, further comprising at least one filler.

10. The composition according to claim 9, wherein the filler is present in a content ranging from 0.1% to 50% by weight relative to the total weight of the composition.

11. The composition according to claim 1, further comprising a liquid fatty phase in a content of less than or equal to 10% by weight relative to the total weight of the composition.

12. The composition according to claim 1, further comprising at least one ingredient chosen from preserving agents, cosmetic active agents, moisturizers, UV-screening agents, thickeners, surfactants and fragrances.

13. The composition according to claim 1, wherein said composition is an eyeshadow.

14. The composition according to claim 2, wherein said composition is an eyeshadow.

15. The composition according to claim 3, wherein said composition is an eyeshadow.

16. The composition according to claim 4, wherein said composition is an eyeshadow.

17. The composition according to claim 5, wherein said composition is an eyeshadow.

18. The composition according to claim 6, wherein said composition is an eyeshadow.

19. The composition according to claim 1, wherein the colouring agent comprises at least one of iron oxide, titanium oxide, zirconium oxide, cerium oxide, zinc oxide, chromium oxide, ferric blue, manganese violet, ultramarine blue, pink or violet, chromium hydrate, chromium hydroxide and bismuth oxychloride.

20. A process for making up an eyelid, comprising applying the composition according to claim 13 to the eyelids.
